(19) United States
(12) Patent Application Publication
(10) Pub. No.: US 2023/0230263 A1
(43) Pub. Date: Jul. 20, 2023

(54) TWO-DIMENSIONAL IMAGE REGISTRATION

(71) Applicant: Auris Health, Inc., Redwood City, CA (US)

(72) Inventors: Elif AYVALI, Redwood City, CA (US); Bulat Ibragimov, Coppenhagen (DK)

(73) Assignee: Auris Health, Inc., Redwood City, CA (US)

(21) Appl. No.: 18/067,691

(22) Filed: Dec. 16, 2022

Related U.S. Application Data
(60) Provisional application No. 63/295,516, filed on Dec. 31, 2021.

(51) Int. Cl. G06T 7/37; G06V 10/20; G06V 10/26; G06T 7/33; A61B 34/20

(52) U.S. Cl. CPC G06T 7/37; A61B 34/20; G06T 7/33; G06V 10/26; G06V 10/255; A61B 2034/2048; A61B 2034/2051; A61B 2034/2061; G06T 2207/10064; G06T 2207/30084; G06V 2201/031; G06V 2201/034

(57) ABSTRACT

The present disclosure relates to systems, devices, and methods to augment a two-dimensional image.

### RELATED APPLICATION(S)

This application claims priority to U.S. Provisional Application No. 63/295,516, filed Dec. 31, 2021, entitled TWO-DIMENSIONAL IMAGE REGISTRATION, the disclosure of which is hereby incorporated by reference in its entirety.

### BACKGROUND

Various medical procedures involve the use of one or more devices configured to penetrate the human anatomy to reach a treatment site. Certain operational processes can involve localizing a medical instrument within the patient and visualizing an area of interest within the patient. To do so, many medical instruments may include sensors to track the location of the instrument and may include vision capabilities, such as embedded cameras or the compatible use with vision probes.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 is a block diagram that illustrates an example two-dimensional image registration system for performing various medical procedures in accordance with aspects of the present disclosure.

FIG. 2 is a diagram illustrating an augmented two-dimensional image in accordance with one or more embodiments.

FIG. 3 is a flow-chart illustrating a method to add positional information of an instrument to a two-dimensional image, according to an example embodiment.

FIG. 4 is a diagram illustrating an example of a two-dimensional image with segmentation data, according to an example embodiment.

FIG. 5 is a diagram illustrating an example augmented non-contrasted image, according to an example embodiment.

### DETAILED DESCRIPTION

The headings provided herein are for convenience only and do not necessarily affect the scope or meaning of disclosure. Although certain exemplary embodiments are disclosed below, the subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

The present disclosure relates to systems, devices, and methods to augment a two-dimensional image with three-dimensional data from a location sensor or any other suitable three-dimensional system data, such as robotic data (e.g., insertion commands, retraction commands, articulation, and the like).

### Two-Dimensional Image Registration System

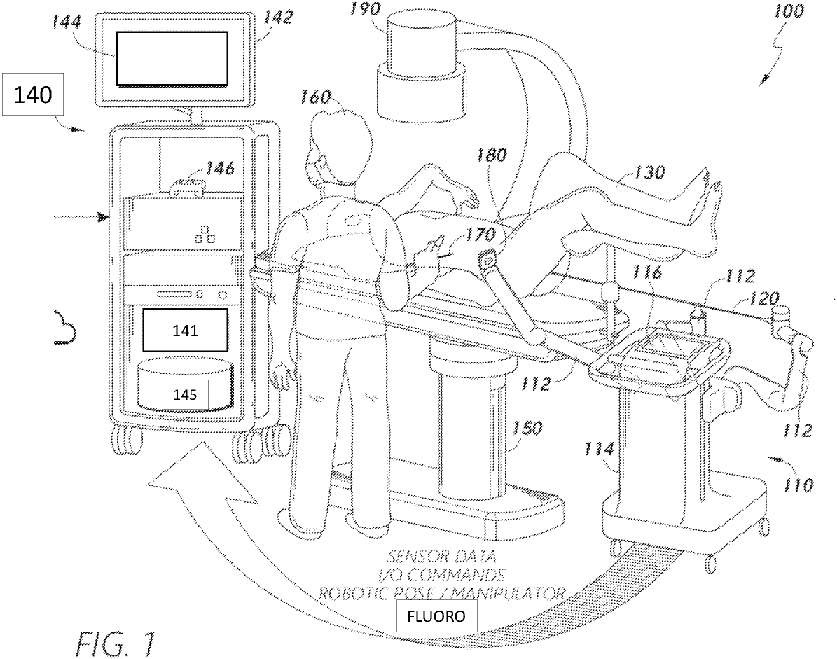

FIG. 1 is a block diagram that illustrates an example two-dimensional image registration system 100 for performing various medical procedures in accordance with aspects of the present disclosure. The two-dimensional image registration system 100 includes a robotic system 110 configured to engage with and/or control a medical instrument 120 to perform a procedure on a patient 130. The two-dimensional image registration system 100 also includes a control system 140 configured to interface with the robotic system 110, provide information regarding the procedure, and/or perform a variety of other operations. For example, the control system 140 can include a display(s) 142 to present certain information to assist the physician 160. The display(s) 142 may be a monitor, screen, television, virtual reality hardware, augmented reality hardware, three-dimensional imaging devices (e.g., hologram devices) and the like, or combinations thereof. The two-dimensional image registration system 100 can include a table 150 configured to hold the patient 130. The system 100 can further include an electromagnetic (EM) field generator 180, which can be held by one or more robotic arms 112 of the robotic system 110 or can be a stand-alone device. In examples, the two-dimensional image registration system 100 can also include an imaging device 190 which can be integrated into a C-arm and/or configured to provide imaging during a procedure, such as for a fluoroscopy-type procedure.

In some implementations, the two-dimensional image registration system 100 can be used to perform a percutaneous procedure. For example, if the patient 130 has a kidney stone that is too large to be removed through a urinary tract, the physician 160 can perform a procedure to remove the kidney stone through a percutaneous access point on the patient 130. To illustrate, the physician 160 can interact with the control system 140 to control the robotic system 110 to advance and navigate the medical instrument 120 (e.g., a scope) from the urethra, through the bladder, up the ureter, and into the kidney where the stone is located. The control system 140 can provide information via the display(s) 142 regarding the medical instrument 120 to assist the physician 160 in navigating the medical instrument 120, such as real-time images captured therewith.

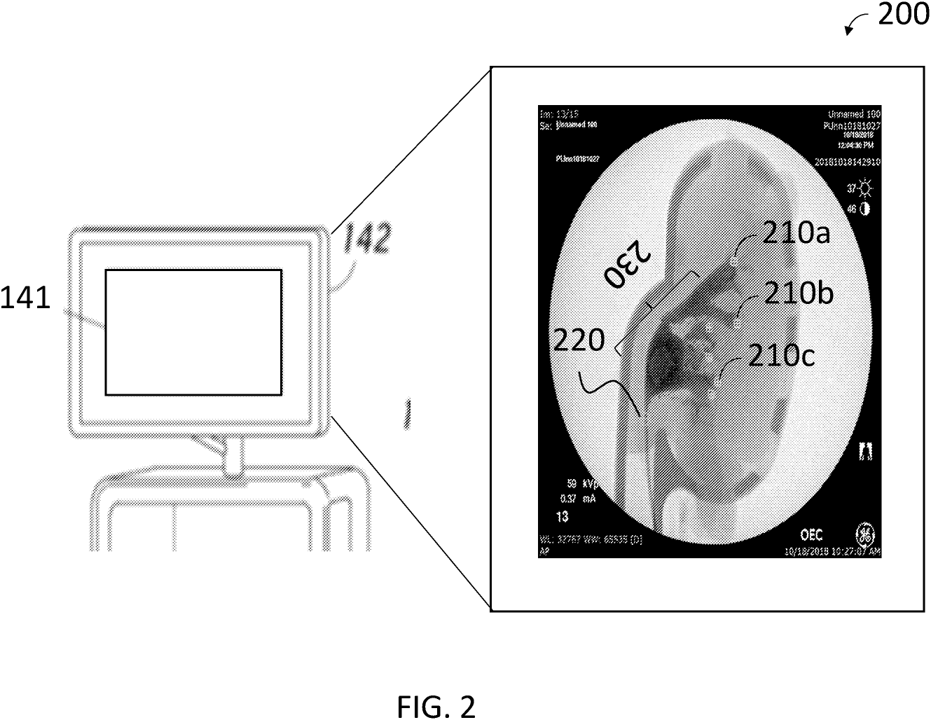

Once at the site of the kidney stone (e.g., within a calyx of the kidney), the medical instrument 120 can be used to designate/tag a target location for the medical instrument 170 (e.g., a needle) to access the kidney percutaneously (e.g., a desired point to access the kidney). To minimize damage to the kidney and/or the surrounding anatomy, the physician 160 can designate a particular papilla as the target location for entering into the kidney with the medical instrument 170. However, other target locations can be designated or determined. To assist the physician in driving the medical instrument 170 into the patient 130 through the particular papilla, the control system 140 can provide a visualization interface 144, which can include a rendering of a two-dimensional image data, augmented based on three-dimensional data from the system, such as location sensor data, robot data, image data, and the like. As is explained in greater detail, the visualization interface 144 may provide information to the operator that is helpful in driving the medical instrument 170 to the target location. An example of a visualization interface 200 is shown in FIG. 2, according to an embodiment. For example, the visualization interface 200 may include visual indicators that map locations of the instrument to locations in the two-dimensional image. For example, an operator may “tag” or otherwise mark or designate a particular location of the instrument as a location of interest. For example, in kidney stone removal, the operator may move the endoscope to different calyxes and tag those locations when the endoscope reaches a calyx of interest. The visualization interface 200 includes tagged locations 210a,b,c. The visualization interface may also render a visual indicator representing the current location of the endoscope, such as by current location indicator 220. Still yet, it may be useful for an operator to understand where the scope has travelled over the course of a procedure. This is shown my trace indicators 230 which provide visual indicators of where the endoscope has been with respect to the anatomy. It is to be appreciated the placement of the locations of the scope within the two-dimensional image may use the registrations techniques described herein to map locations in a location sensor space to a location within a two-dimensional image.

With continued reference to FIG. 1, once the instrument 170 has reached the target, the physician 160 can use the medical instrument 170 and/or another medical instrument to extract the kidney stone from the patient 130. One such instrument may be a percutaneous catheter. The percutaneous catheter may be an instrument with steering capabilities, much like the instrument 120, but may, in some embodiments, lack a dedicated camera or location sensor. Some embodiments may use the augmented visualization interface 144 to render augmented images that are helpful in driving the percutaneous catheter within the anatomy.

Although the above percutaneous procedure and/or other procedures are discussed in the context of using the medical instrument 120, in some implementations a percutaneous procedure can be performed without the assistance of the medical instrument 120. Further, the two-dimensional image registration system 100 can be used to perform a variety of other procedures.

Moreover, although many embodiments describe the physician 160 using the medical instrument 170, the medical instrument 170 can alternatively be used by a component of the two-dimensional image registration system 100. For example, the medical instrument 170 can be held/manipulated by the robotic system 110 (e.g., the one or more robotic arms 112) and the techniques discussed herein can be implemented to control the robotic system 110 to insert the medical instrument 170 with the appropriate pose (or aspect of a pose, such as orientation or position) to reach a target location.

In the example of FIG. 1, the medical instrument 120 is implemented as a scope and the medical instrument 170 is implemented as a needle. Thus, for ease of discussion, the medical instrument 120 is referred to as “the scope 120” or “the lumen-based medical instrument 120,” and the medical instrument 170 is referred to as “the needle 170” or “the percutaneous medical instrument 170.” However, the medical instrument 120 and the medical instrument 170 can each be implemented as a suitable type of medical instrument including, for example, a scope (sometimes referred to as an “endoscope”), a needle, a catheter, a guidewire, a lithotripter, a basket retrieval device, forceps, a vacuum, a needle, a scalpel, an imaging probe, jaws, scissors, graspers, needle holder, micro dissector, staple applier, tacker, suction/irrigation tool, clip applier, and so on. In some embodiments, a medical instrument is a steerable device, while other embodiments a medical instrument is a non-steerable device. In some embodiments, a surgical tool refers to a device that is configured to puncture or to be inserted through the human anatomy, such as a needle, a scalpel, a guidewire, and so on. However, a surgical tool can refer to other types of medical instruments.

In some embodiments, a medical instrument, such as the scope 120 and/or the needle 170, includes a sensor that is configured to generate sensor data, which can be sent to another device. In examples, sensor data can indicate a location/orientation of the medical instrument and/or can be used to determine a location/orientation of the medical instrument. For instance, a sensor can include an electromagnetic (EM) sensor with a coil of conductive material. Here, an EM field generator, such as the EM field generator 180, can provide an EM field that is detected by the EM sensor on the medical instrument. The magnetic field can induce small currents in coils of the EM sensor, which can be analyzed to determine a distance and/or angle/orientation between the EM sensor and the EM field generator. Further, a medical instrument can include other types of sensors configured to generate sensor data, such as one or more of any of: a camera, a range sensor, a radar device, a shape sensing fiber, an accelerometer, a gyroscope, a satellite-based positioning sensor (e.g., a global positioning system (GPS)), a radio-frequency transceiver, and so on. In some embodiments, a sensor is positioned on a distal end of a medical instrument, while in other embodiments a sensor is positioned at another location on the medical instrument. In some embodiments, a sensor on a medical instrument can provide sensor data to the control system 140 and the control system 140 can perform one or more localization techniques to determine/track a position and/or an orientation of a medical instrument.

In some embodiments, the two-dimensional image registration system 100 may record or otherwise track the runtime data that is generated during a medical procedure. This runtime data may be referred to as system data. For example, the two-dimensional image registration system 100 may track or otherwise record the sensor readings (e.g., sensor data) from the instruments (e.g., the scope 120 and the needle 170) in data store 145A (e.g., a computer storage system, such as computer readable memory, database, filesystem, and the like). In addition to sensor data, the two-dimensional image registration system 100 can store other types of system data in data store 145. For example, in the context of FIG. 1, the system data can further include time series data of the video images captured by the scope 120, status of the robotic system 110, commanded data from an I/O device(s) (e.g., I/O device(s) 146 discussed below), audio data (e.g., as may be captured by audio capturing devices embedded in the two-dimensional image registration system 100, such as microphones on the medical instruments, robotic arms, or elsewhere in the two-dimensional image registration system), external (relative to the patient) imaging device (such as RGB cameras, LIDAR imaging sensors, fluoroscope imaging sensors, etc.), and image data from the imaging device 190, and the like.

As shown in FIG. 1, the control system 140 includes an augmentation module 141 which may be control circuitry configured to operate on the system data and the two-dimensional image data stored in the case data store 145 to generate an augmented representation of the two-dimensional image data with three-dimensional system data. As is discussed in greater detail below, the augmentation module 141 may employ machine learning techniques to segment two-dimensional image data according to the anatomy or instruments present in the two-dimensional images. In some embodiments, once the two-dimensional image data has been segmented, the augmentation module 141 may augment two-dimensional image data with three-dimensional data from the system 100.

The term “scope” or “endoscope” are used herein according to their broad and ordinary meanings and can refer to any type of elongate medical instrument having image generating, viewing, and/or capturing functionality and configured to be introduced into any type of organ, cavity, lumen, chamber, and/or space of a body. For example, references herein to scopes or endoscopes can refer to a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), a borescope, and so on.

A scope can comprise a tubular and/or flexible medical instrument that is configured to be inserted into the anatomy of a patient to capture images of the anatomy. In some embodiments, a scope can accommodate wires and/or optical fibers to transfer signals to/from an optical assembly and a distal end of the scope, which can include an imaging device, such as an optical camera. The camera/imaging device can be used to capture images of an internal anatomical space, such as a target calyx/papilla of a kidney. A scope can further be configured to accommodate optical fibers to carry light from proximately-located light sources, such as light-emitting diodes, to the distal end of the scope. The distal end of the scope can include ports for light sources to illuminate an anatomical space when using the camera/imaging device. In some embodiments, the scope is configured to be controlled by a robotic system, such as the robotic system 110. The imaging device can comprise an optical fiber, fiber array, and/or lens. The optical components can move along with the tip of the scope such that movement of the tip of the scope results in changes to the images captured by the imaging device.

A scope can be articulable, such as with respect to at least a distal portion of the scope, so that the scope can be steered within the human anatomy. In some embodiments, a scope is configured to be articulated with, for example, five or six degrees of freedom, including X, Y, Z coordinate movement, as well as pitch, yaw, and roll. A position sensor(s) of the scope can likewise have similar degrees of freedom with respect to the position information they produce/provide. A scope can include telescoping parts, such as an inner leader portion and an outer sheath portion, which can be manipulated to telescopically extend the scope. A scope, in some instances, can comprise a rigid or flexible tube, and can be dimensioned to be passed within an outer sheath, catheter, introducer, or other lumen-type device, or can be used without such devices. In some embodiments, a scope includes a working channel for deploying medical instruments (e.g., lithotripters, basketing devices, forceps, etc.), irrigation, and/or aspiration to an operative region at a distal end of the scope.

The robotic system 110 can be configured to at least partly facilitate execution of a medical procedure. The robotic system 110 can be arranged in a variety of ways depending on the particular procedure. The robotic system 110 can include the one or more robotic arms 112 configured to engage with and/or control the scope 120 to perform a procedure. As shown, each robotic arm 112 can include multiple arm segments coupled to joints, which can provide multiple degrees of movement. In the example of FIG. 1, the robotic system 110 is positioned proximate to the patient’s 130 legs and the robotic arms 112 are actuated to engage with and position the scope 120 for access into an access point, such as the urethra of the patient 130. When the robotic system 110 is properly positioned, the scope 120 can be inserted into the patient 130 robotically using the robotic arms 112, manually by the physician 160, or a combination thereof. The robotic arms 112 can also be connected to the EM field generator 180, which can be positioned near a treatment site, such as within proximity to the kidneys of the patient 130.

The robotic system 110 can also include a support structure 114 coupled to the one or more robotic arms 112. The support structure 114 can include control electronics/circuitry, one or more power sources, one or more pneumatics, one or more optical sources, one or more actuators (e.g., motors to move the one or more robotic arms 112), memory/data storage, and/or one or more communication interfaces. In some embodiments, the support structure 114 includes an input/output (I/O) device(s) 116 configured to receive input, such as user input to control the robotic system 110, and/or provide output, such as a graphical user interface (GUI), information regarding the robotic system 110, information regarding a procedure, and so on. The I/O device(s) 116 can include a display, a touchscreen, a touchpad, a projector, a mouse, a keyboard, a microphone, a speaker, etc. In some embodiments, the robotic system 110 is movable (e.g., the support structure 114 includes wheels) so that the robotic system 110 can be positioned in a location that is appropriate or desired for a procedure. In other embodiments, the robotic system 110 is a stationary system. Further, in some embodiments, the robotic system 112 is integrated into the table 150.

The robotic system 110 can be coupled to any component of the two-dimensional image registration system 100, such as the control system 140, the table 150, the EM field generator 180, the scope 120, and/or the needle 170. In some embodiments, the robotic system is communicatively coupled to the control system 140. In one example, the robotic system 110 can be configured to receive a control signal from the control system 140 to perform an operation, such as to position a robotic arm 112 in a particular manner, manipulate the scope 120, and so on. In response, the robotic system 110 can control a component of the robotic system 110 to perform the operation. In another example, the robotic system 110 is configured to receive an image from the scope 120 depicting internal anatomy of the patient 130 and/or send the image to the control system 140, which can then be displayed on the display(s) 142. Furthermore, in some embodiments, the robotic system 110 is coupled to a component of the two-dimensional image registration system 100, such as the control system 140, in such a manner as to allow for fluids, optics, power, or the like to be received therefrom. Example details of the robotic system 110 are discussed in further detail below in reference to FIG. 12.

The control system 140 can be configured to provide various functionality to assist in performing a medical procedure. In some embodiments, the control system 140 can be coupled to the robotic system 110 and operate in cooperation with the robotic system 110 to perform a medical procedure on the patient 130. For example, the control system 140 can communicate with the robotic system 110 via a wireless or wired connection (e.g., to control the robotic system 110 and/or the scope 120, receive an image(s) captured by the scope 120, etc.), provide fluids to the robotic system 110 via one or more fluid channels, provide power to the robotic system 110 via one or more electrical connections, provide optics to the robotic system 110 via one or more optical fibers or other components, and so on. Further, in some embodiments, the control system 140 can communicate with the needle 170 and/or the scope 170 to receive sensor data from the needle 170 and/or the endoscope 120 (via the robotic system 110 and/or directly from the needle 170 and/or the endoscope 120). Moreover, in some embodiments, the control system 140 can communicate with the table 150 to position the table 150 in a particular orientation or otherwise control the table 150. Further, in some embodiments, the control system 140 can communicate with the EM field generator 180 to control generation of an EM field around the patient 130.

The control system 140 includes various I/O devices configured to assist the physician 160 or others in performing a medical procedure. In this example, the control system 140 includes an I/O device(s) 146 that is employed by the physician 160 or other user to control the scope 120, such as to navigate the scope 120 within the patient 130. For example, the physician 160 can provide input via the I/O device(s) 146 and, in response, the control system 140 can send control signals to the robotic system 110 to manipulate the scope 120. Although the I/O device(s) 146 is illustrated as a controller in the example of FIG. 1, the I/O device(s) 146 can be implemented as a variety of types of I/O devices, such as a touchscreen, a touch pad, a mouse, a keyboard, a surgeon or physician console, virtual reality hardware, augmented hardware, microphone, speakers, haptic devices, and the like.

As also shown in FIG. 1, the control system 140 can include the display(s) 142 to provide various information regarding a procedure. As noted above, the display(s) 142 can present the visualization interface 144 to assist the physician 160 in the percutaneous access procedure (e.g., manipulating the needle 170 towards a target site). The display(s) 142 can also provide (e.g., via the visualization interface 144 and/or another interface) information regarding the scope 120. For example, the control system 140 can receive real-time images that are captured by the scope 120 and display the real-time images via the display(s) 142. Additionally or alternatively, the control system 140 can receive signals (e.g., analog, digital, electrical, acoustic/sonic, pneumatic, tactile, hydraulic, etc.) from a medical monitor and/or a sensor associated with the patient 130, and the display(s) 142 can present information regarding the health or environment of the patient 130. Such information can include information that is displayed via a medical monitor including, for example, a heart rate (e.g., ECG, HRV, etc.), blood pressure/rate, muscle bio-signals (e.g., EMG), body temperature, blood oxygen saturation (e.g., SpO2), CO2, brainwaves (e.g., EEG), environmental and/or local or core body temperature, and so on.

To facilitate the functionality of the control system 140, the control system 140 can include various components (sometimes referred to as “subsystems”). For example, the control system 140 can include control electronics/circuitry, as well as one or more power sources, pneumatics, optical sources, actuators, memory/data storage devices, and/or communication interfaces. In some embodiments, the control system 140 includes control circuitry comprising a computer-based control system that is configured to store executable instructions, that when executed, cause various operations to be implemented. In some embodiments, the control system 140 is movable, such as that shown in FIG. 1, while in other embodiments, the control system 140 is a stationary system. Although various functionality and components are discussed as being implemented by the control system 140, any of this functionality and/or components can be integrated into and/or performed by other systems and/or devices, such as the robotic system 110, the table 150, and/or the EM generator 180 (or even the scope 120 and/or the needle 170). Example details of the control system 140 are discussed in further detail below in reference to FIG. 13.

The imaging device 190 can be configured to capture/generate one or more images of the patient 130 during a procedure, such as one or more x-ray or CT images. In examples, images from the imaging device 190 can be provided in real-time to view anatomy and/or medical instruments, such as the scope 120 and/or the needle 170, within the patient 130 to assist the physician 160 in performing a procedure. The imaging device 190 can be used to perform a fluoroscopy (e.g., with a contrast dye within the patient 130) or another type of imaging technique.

The various components of the two-dimensional image registration system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, etc. Further, in some embodiments, the components of the two-dimensional image registration system 100 are connected for data communication, fluid/gas exchange, power exchange, and so on, via one or more support cables, tubes, or the like.

Although various techniques and systems are discussed as being implemented as robotically-assisted procedures (e.g., procedures that at least partly use the two-dimensional image registration system 100), the techniques and systems can be implemented in other procedures, such as in fully-robotic medical procedures, human-only procedures (e.g., free of robotic systems), and so on. For example, the two-dimensional image registration system 100 can be used to perform a procedure without a physician holding/manipulating a medical instrument (e.g., a fully-robotic procedure). That is, medical instruments that are used during a procedure, such as the scope 120 and the needle 170, can each be held/controlled by components of the two-dimensional image registration system 100, such as the robotic arm(s) 112 of the robotic system 110.

### Two-Dimensional Image Registration Methods and Operations

Details of the methods and operations of exemplary two-dimensional image registration systems are now discussed. The methods and operation disclosed herein are described relative to the two-dimensional image registration system 100 shown in FIG. 1. However, it is to be appreciated that the methods and operations may be performed by any of the components, alone or in combination, discussed herein.

As discussed above, a two-dimensional image registration system may register a coordinate frame of a two-dimensional image (e.g., an image acquired from a fluoroscope) with three-dimensional data of a robotic system. In some cases, the two-dimensional image used in the registration may include additional information that is difficult or desirable to acquire in later stages of a medical procedure. This may include a fluoroscope image acquired with a contrast agent to identify anatomy that is not visible or is at least less visible with a non-contrast fluoroscopy image. Once registered, a two-dimensional image registration system may render the two-dimensional image with information derived from the three-dimensional data of the robotic system. In this way, the two-dimensional image registration system may be able to provide an operator a registered two-dimensional image with information regarding a current location of an instrument, even where there is a difference in time from when the two-dimensional image was acquired and when the location of the instrument is determined.

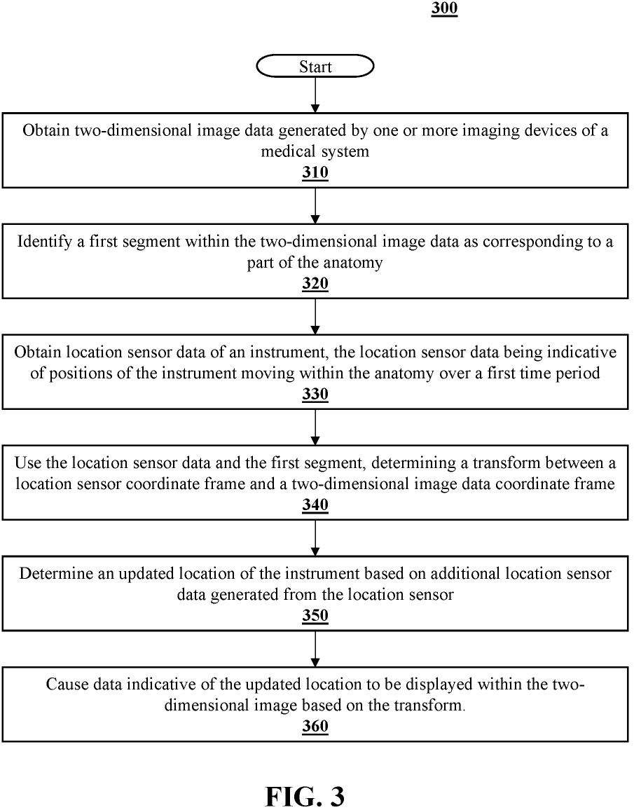

FIG. 3 is a flow-chart illustrating a method 300 to add positional information of an instrument to a two-dimensional image, according to an example embodiment. As used herein, “positional information” may refer to any suitable component or combination of a pose, such as a location or orientation. As FIG. 3 shows, the method 300 may begin at block 310, where the two-dimensional image registration system obtains two-dimensional image data generated by one or more imaging devices of a two-dimensional image registration system. For example, during a medical procedure, a fluoroscope may capture an image that includes a representation of a patient’s anatomy. In some cases, a contrast agent may be introduced into the patient to increase the details of the anatomy picked up by the fluoroscope. Such may be the case with a pyelogram to allow the fluoroscope to obtain the internal morphology of the kidney, such as a ureter, a renal pelvis, or a calyx.

At block 320, the two-dimensional image registration system may identify a first segment within the two-dimensional image data as corresponding to a part of the anatomy. As merely an example, and not a limitation, where the two-dimensional image is a pyelogram, the part of the anatomy may correspond to at least one of: a ureter, a renal pelvis, or a calyx. However, the part of anatomy can refer to any suitable part of anatomy for anatomies other than a kidney.

It is to be appreciated that, at block 320, the two-dimensional image registration system may identify additional segments besides the first segment. For example, the two-dimensional image registration system may identify multiple segments within a pyelogram, where each of the multiple segments corresponds to a different part of the anatomy, such as a ureter, a renal pelvis, or a calyx.

The two-dimensional image registration system may identify the segments from the two-dimensional image data according to various techniques. For example, in one embodiment, the two-dimensional image registration system may utilize a neural network (e.g., a convolutional neural network with a U-net architecture) that has learned the consistent intensity patterns that define the anatomy of interest. As another example, the two-dimensional image registration system may provide a user interface that receives user input on the boundaries for the parts of the anatomy. Such user inputs may, in some cases, define the boundaries themselves or, in other cases, may correct or otherwise modify segments automatically generated by the two-dimensional image registration system based on a neural network approach.

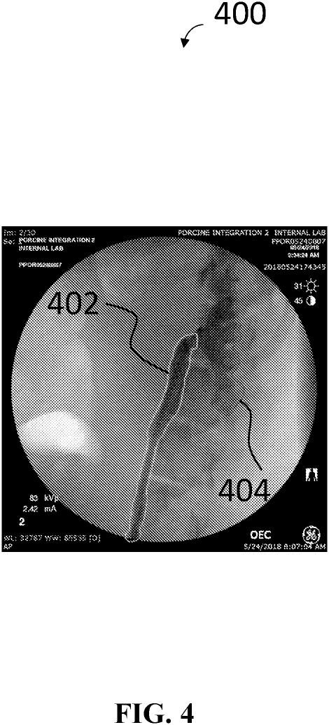

FIG. 4 is a diagram illustrating an example of a two-dimensional image with segmentation data 400, according to an example embodiment. The segmentation data may include a segmentation 402 to identify the renal ureter and a segmentation 404 to identify a renal pelvis and calyxes.

With reference back to FIG. 3, at block 330, the two-dimensional image registration system may obtain location sensor data of an instrument. The location sensor data may be indicative of positions of an instrument moving within the anatomy over a first time period. For example, the location sensor data may be sensor data derived from an EM sensor, a shape sensing fiber, an accelerometer, a magnetometer, a gyroscope, or the like. It is to be appreciated that such location sensor data may be expressed according to a coordinate frame different than a coordinate frame of the imaging device. In context of block 330, the first time period may be when the system first begins collecting location data to the acquisition of the two-dimensional image data. In another example, the first time period may correspond to a time period in which the operator drives and “tags” specific anatomy features.

In some embodiments, the two-dimensional image registration system may obtain additional data regarding the location of the instrument. For example, the two-dimensional image registration system may obtain robotic data (e.g., kinematic data derived from commanded movement of the robotic arms). Alternatively or additionally, the two-dimensional image registration system may obtain tagged data. Tagged data may refer to automatic/user-initiated data that designates a particular position in location sensor space with a determinable anatomy. For example, an operator may drive an instrument scope to touch a particular calyx and, responsive to an input from the operator, the system may tag the location identified in the location sensor space as the particular calyx.

At block 340, the two-dimensional image registration system determines a transform between a location sensor coordinate frame and a two-dimensional image data coordinate frame using the location sensor data and the first segment. As used herein, a transform may be data or logic that maps one coordinate frame to another. In the case of block 340, the transform may map the locations from the location sensor coordinate frame to a two-dimensional image data coordinate frame. Once the two-dimensional image registration system completes block 340 and the coordinate frames for the location sensor and the two-dimensional images are registered to each other, the two-dimensional image registration system may map locations from the instruments to the two-dimensional image.

Although discussed in greater detail below, an initial step for automated registration is moving a two-dimensional image and three-dimensional location sensor data into the same dimension. To move two-dimensional data into three-dimension, the system 100 may add a dummy dimension to the two-dimensional image, and the problem turns into aligning three-dimensional location sensor data with a three-dimensional plane representing the two-dimensional image.

To move the location sensor data into two-dimension, the system determines the angle from which the two-dimensional image was taken. One solution is to get the angle explicitly from the imaging device. An alternative solution is to assume that the two-dimensional image was taken from the “visibility” angle, so we also orient the location sensor data to the “visibility” angle, i.e. orient EM points according to their eigenvalues. Once the registration dimension is unified, location sensor data are registered to the two-dimensional image by combining AI-based pyelogram annotation, rigid and non-rigid alignment of the 3D point clouds (e.g., coherent point drift), image filters for the enhancment and segmentation of tubular structures (e.g., Frangi filter algorithm), and any other suitable techniques.

In terms of determining a “visibility” angle, system may use any number of techniques. For example, some systems may look at the CT preoperatively to see the angulation of the kidney plane with respect to the bed. Assuming the angulation is 10 degree anterior. In modified-supine, the patient may be tilted 15 degrees to expose the flank. The visibility angle may then be a function of those, say, 10+15=25 degree. Other system may instead find the principal axes in the location sensor trace to find the kidney plane (basically fitting a plane to location sensor data). The system may know where bed is with respect to the location sensor space (cart parallel to the bed, robot is holding to CFG).

Once the location sensor coordinate frame and the two-dimensional image coordinate frame are registered, the two-dimensional image registration system may begin augmenting the two-dimensional image with information regarding the location of the instrument. For example, at block 350, the two-dimensional image registration system may determine an updated location of the instrument based on additional location sensor data generated from the location sensor. In some embodiments, the additional location sensor data may be generated at a time period after the first time period and may refer to a most recent time period.

At block 360, the two-dimensional image registration system may cause data indicative of the updated location to be displayed within the two-dimensional image based on the transform generated at block 340. As just discussed, the updated location may refer to a location of the instrument after the coordinate frames of the location sensor and the two-dimensional image are registered. In some cases, the updated location may refer to the most recent location of the instrument. Thus, at the conclusion of block 360, the two-dimensional image registration system has augmented the two-dimensional image with a current location data of the instrument. This may be beneficial as the two-dimensional image may include additional details (e.g., contrast dye) that would not be normally present if the two-dimensional image was to be retaken at a time coinciding with when the instrument is at the updated location. Some embodiments may display the data indicative of the updated location as a model of the instrument. Other embodiments may display the data indicative of the updated location as an icon that represents positional information, such a location and/orientation.

It is to be appreciated that locations other than the updated location may be displayed within the two-dimensional image based on the transform generated at block 340. For example, some embodiments of the two-dimensional image registration system may cause historical locations of the instrument to be displayed within the two-dimensional image based on the transform generated at block 340. For example, once the two-dimensional image registration system generates the transform at block 340, the two-dimensional image registration system may cause some or all of the location sensor data obtained at block 330 to be displayed within the two-dimensional image. In this way, the historical path of the instrument through the anatomy can be represented within the two-dimensional image.

The two-dimensional image registration system may represent the location data within the two-dimensional image using various ways graphical icons. For example, the two-dimensional image registration system may represent the location data as discrete graphical icons, such as dots, squares, arrows, or any other graphical icon, spaced out, in some cases, according to a frequency, such that graphical icons spaced closer together represent an instrument moving along a path at a slower rate. Additional or alternatively, the two-dimensional image registration system may represent the location data as lines to designate a path. In any of these embodiments, the two-dimensional image registration system may represent use different properties to distinguish different aspects of a procedure. For example, the two-dimensional image registration system may designate a first instrument using a first type of graphical icon and a second instrument using a second but different type of graphical icon. Additionally, the two-dimensional image registration system may use one type of graphical icon to represent a path of an instrument and another graphical icon to represent a user or system driven event, such as a user tagging an anatomy, an instrument being in a particular state (e.g., lasing, biopsy acquisition, delivery of therapeutics), and the like.

FIG. 2 provides an example embodiment illustrating one type of graphical icon (e.g., dots) representing a path of an instrument and another type of graphical icon (e.g., cross-hairs) representing calyxes tagged by an operator.

### Registration

The method 300 at block 340 discusses generating a transform between a location sensor coordinate frame and a two-dimensional image data coordinate frame using the location sensor data and the first segment. This process may be referred to as registration. Example embodiments of registration are now discussed in greater detail. In the context of embodiments discussed herein, registration may be defined as aligning three-dimensional location sensor data with two-dimensional fluoro image data. There can be several steps involved in registration, such as: (1) segmentation of an anatomy depicted in a two-dimensional image; (2) generation of an anatomy level set from the segmentation results; and (3) alignment of the three-dimensional location sensor data with the two-dimensional image using a level set-based distance map. To simplify the discussion of these steps, embodiments are discussed in the context of kidney anatomy, but it is to be appreciated that any suitable anatomy may be segmented using these approaches. Further, the discussion below focuses on location sensor data but other embodiments may include any additional system data for identifying the location and positioning of an instrument, such as robotic data and the like.

### I. Segmentation

Segmentation of a kidney tissues can be based on machine learning methods employed by the two-dimensional image registration system. A database of kidney fluoros with contrast is collected and manually annotated with all tissues of interest. These fluoro images are normalized to compensate for intensity fluctuations, noise, different resolutions, etc. An encoder-decoder neural network designed for pixelwise image segmentation, referred herein as a “segmentation network,” is trained on the normalized images from the database to learn the appearance of the kidney tissues.

A new fluoro will be acquired as preparation for the percutaneous nephropathy procedure. This fluoro image will be normalized and then processed by the previously trained segmentation network. The segmentation network results will be the masks of the ureter, kidney pelvis, and calyces generated for the new fluoro image. The resulting segmentation masks will be of the same size as the new fluoro.

### II. Level Set

A kidney level set will be generated from the kidney tissue segmentation. All pixels that correspond to the outer borders of kidney tissue will have a value of zero on the level set. All pixels outside segmented kidney tissues will have negative values that encode the negative distance to the closest pixel that belongs to the kidney segmentation border. All the pixels that are located inside kidney tissue segmentation will have positive values that encode the distance to the closest pixel that belongs to the kidney segmentation border. The “deeper inside” the kidney a pixel is, the higher its value in the level set is.

### III. Alignment

Alignment of the three-dimensional sensor data with the two-dimensional fluoro uses the kidney level set. Our aim is to position three-dimensional sensor data in such a way that sensor coordinate points shall pass through the pixels of the level set with the highest total sum. To achieve this, the two-dimensional image registration system may execute a number of steps that include the following:

A. The two-dimensional image registration system may start with an initial guess where the sensor is currently located inside the kidney. There are a number of ways this initial guess can be achieved. For example, the two-dimensional image registration system can set the initial guess to a known position in the anatomy, such as the lowest point of the ureter on fluoro. Alternatively, the two-dimensional image registration system can instruct the operator to position the instrument to a known position within the anatomy.

B. The two-dimensional image registration system may then define a set of acceptable transformations. The acceptable translations of the sensor data over the two-dimensional image can be unlimited. The acceptable in-plane and out-of-plane rotations of the sensor data is limited using the standard positioning of the patient, robot, and fluoroscope information. Note the limited rotation does not mean that the three-dimensional sensor data cannot rotate but rather that two-dimensional image registration system may have some restrictions on the possible rotations so that three-dimensional sensor data will not turn by 180 degrees during this procedure. The scaling is also limited by the standard positioning of the depicted objects.

Using the initial guess, the two-dimensional image registration system projects three-dimensional sensor data to the two-dimensional fluoro, i.e. remove the dimension that is oriented along the fluoro normal. The two-dimensional image registration system then computes the total value for all projected sensor points over the kidney level set. The two-dimensional image registration system then updates the initial guess according to the acceptable transformations to improve the positioning of the projected three-dimensional points. The updating can be performed using a gradient descent algorithm that maximizes the total value for all projected sensor points.

### Augmenting Subsequent Two-Dimensional Images

Generating segmentation data relating to an anatomy from a contrast two-dimensional image may have additional applications for a procedure. For example, the two-dimensional image registration system may acquire two-dimensional images later in the procedure, but these subsequent two-dimensional images may lack details of the anatomy found in the segmented two-dimensional image because these subsequent two-dimensional images may be taken without administering a contrast agent to the patient. At the same time, such non-contrast two-dimensional images may have the instruments visible. The two-dimensional image registration system may use the segmented anatomy to augment the non-contrast two-dimensional images by superimposing the anatomical details obtained from the anatomy segmentation and the depicted instruments. To do so, the two-dimensional image registration system may: (1) segment the instrument (and component parts, such as scope tip) from a non-contrast image; (2) estimate fluoro anatomical resolution; and (3) register the previously acquired fluoro with contrast to the segmented instrument of the fluoro without contrast.

### I. Instrument Segmentation

Segmentation of the scope and, in some cases, its component parts (e.g., instrument tip) may involve methodologies similar to those discussed above for segmentation of the anatomies. For example, the non-contrast two-dimensional image may be processed by a neural network trained with a database of annotated two-dimensional images that identify instruments in the two-dimensional images.

A result of the segmentation is an instrument mask and the coordinates and orientation of the instrument tip are obtained.

### II. Anatomical Resolution Estimation

To estimate the resolution of the depicted structures in millimeters per pixel, the two-dimensional image registration system first determines a centerline of the instrument segmentation. For points along the centerline of the instrument, the two-dimensional image registration system finds a normal direction, i.e. the direction orthogonal to the centerline. The distance between the most distant points segmented as the instrument along the normal direction is interpreted by the two-dimensional image registration system as the radius of the scope at the centerline point. By computing the radii of the instrument for all centerline points, the two-dimensional image registration system determines the average radius of the instrument, as may be measured in pixels. By normalizing this average radius to the known radius of the instrument, the two-dimensional image registration system determines the anatomical-to-fluoro resolution, i.e. how many millimeters of the kidney tissue are in one pixel. In one embodiment, the two-dimensional image registration system may obtain the known radius of the instrument via a calibration parameter transmitted to the control system when the instrument is docked to a robot arm. In other embodiments, the two-dimensional image registration system may obtain the known radius based on an identification of the instrument received from an operator and a lookup table mapping instruments to properties, such as instrument measurements. In other embodiment, the two-dimensional registration system could compare the size and shape of the instrument tip from the know instrument properties and tip segmentation result. This information can be combined with the radii analysis to improve the accuracy of the resolution estimation.

### III. Registration of the Segmented Data

The segmented instrument in the non-contrast image should fit inside the patient’s anatomy. Considering the tissue elasticity and time elapsed between contrasted fluoro acquisition and non-contrast fluoro with instrument acquisition, the instrument is expected to be positioned as inside as possible the segmented anatomy derived from the contrast image. In some embodiments, the two-dimensional image registration system may take advantage that the possible articulations of the instruments and general shape of the anatomy (e.g., in the context of a kidney, the ureter) to limit the possible positions of the instrument within the anatomy. This positioning is obtained using a simplified version of the algorithm for three-dimensional location sensor to two-dimensional image registration discussed above. The simplification comes from the fact the segmented instrument is already two-dimensional in contrast to three-dimensional location sensor data. The two-dimensional image registration system can use this to limit the acceptable transformations. In particular, the two-dimensional image registration system can restrict scaling based on: 1) restricting out-of-plane rotations; and 2) restricting in-plane rotations based on an assumption that the imaging device has not been moved during the procedure. Based on this, the two-dimensional image registration system may end up with translations and some small scaling and in-plane rotations.

### IV. Rendering Augmented Non-Contrasted Image

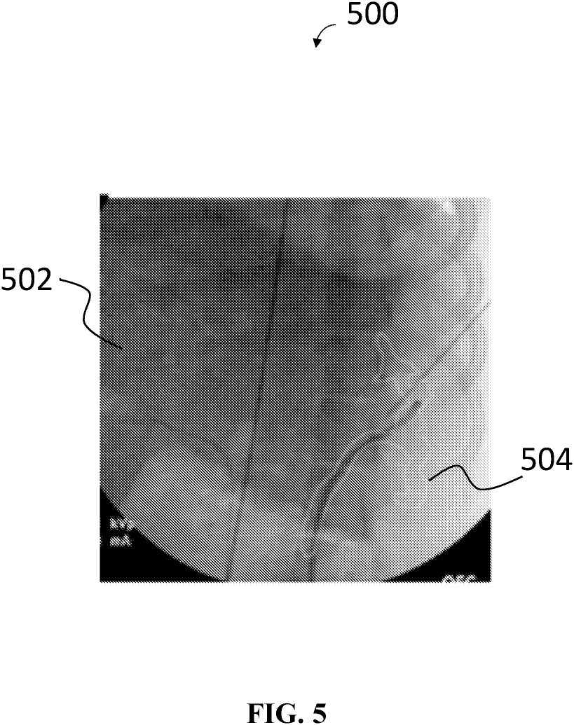

After registering the segmented instrument with the segmented anatomy, the two-dimensional image registration system may augment the non-contrasted two-dimension image with the anatomy segmentation previously acquired. This augmented non-contrast two-dimension image is then rendered on a display device for an operator of the two-dimensional image registration system. The augmented non-contrasted two-dimensional image shows the operator where the borders of the anatomy tissues are located with respect to the instrument. Another potential benefit is that the non-contrast fluoro analysis is that two-dimensional image registration system can improve the initial guess for fluoro registration discussed above. FIG. 5 is a diagram illustrating an example augmented non-contrasted image 500, according to an example embodiment. The augmented non-contrasted image 500 may include two-dimensional image data 502 with anatomy segmentation data 504 superimposed onto the two-dimensional image data 502. As discussed, the anatomy segmentation data 504 may be data derived from a contrasted two-dimensional image data, where the system segments the anatomy.

### Implementing Systems and Terminology

Implementations disclosed herein provide systems, methods and apparatus to augment a two-dimensional image. Various implementations described herein provide for improved visualization of a medical instrument or medical instruments performing a medical procedure.

The two-dimensional image registration system 100 can include a variety of other components. For example, the two-dimensional image registration system 100 can include one or more control circuitry, power sources, pneumatics, optical sources, actuators (e.g., motors to move the robotic arms), memory, and/or communication interfaces (e.g., to communicate with another device). In some embodiments, the memory can store computer-executable instructions that, when executed by the control circuitry, cause the control circuitry to perform any of the operations discussed herein. For example, the memory can store computer-executable instructions that, when executed by the control circuitry, cause the control circuitry to receive input and/or a control signal regarding manipulation of the robotic arms and, in response, control the robotic arms to be positioned in a particular arrangement.

The various components of the two-dimensional image registration system 100 can be electrically and/or communicatively coupled using certain connectivity circuitry/devices/features, which can or may not be part of the control circuitry. For example, the connectivity feature(s) can include one or more printed circuit boards configured to facilitate mounting and/or interconnectivity of at least some of the various components/circuitry of the two-dimensional image registration system 100. In some embodiments, two or more of the control circuitry, the data storage/memory, the communication interface, the power supply unit(s), and/or the input/output (I/O) component(s), can be electrically and/or communicatively coupled to each other.

The term “control circuitry” is used herein according to its broad and ordinary meaning, and can refer to any collection of one or more processors, processing circuitry, processing modules/units, chips, dies (e.g., semiconductor dies including come or more active and/or passive devices and/or connectivity circuitry), microprocessors, micro-controllers, digital signal processors, microcomputers, central processing units, graphics processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware state machine (and/or implements a software state machine), analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can be implemented in accordance with embodiments of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. may be either X, Y, or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the disclosure should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

1. A method to augment a two-dimensional image with positional information of an instrument, the method comprising:
obtaining two-dimensional image data generated by one or more imaging devices of a medical system, the two-dimensional image data corresponding to a location sensor coordinate frame;
identifying a first segment of an anatomy within the two-dimensional image data as corresponding to a part of the anatomy, the two-dimensional image data corresponding to a two-dimensional image data coordinate frame;
obtaining location sensor data of the instrument from a location sensor, the location sensor data being indicative of positions of the instrument moving within the anatomy over a first time period;
using the location sensor data and the first segment of the anatomy, determining a transform between the location sensor coordinate frame and the two-dimensional image data coordinate frame;
determining an updated location of the instrument using additional location sensor data generated from the location sensor; and
causing data indicative of the updated location to be displayed within the two-dimensional image using the transform.

2. (canceled)

3. (canceled)

4. (canceled)

5. (canceled)

6. (canceled)

7. (canceled)

8. (canceled)

9. The method of claim 1, wherein determining the transform between the location sensor coordinate frame and the two-dimensional image data coordinate frame further comprises:
generating a kidney map using the location sensor data; and
registering the two-dimensional imaging data and the kidney map.

10. The method of claim 9, further comprising:
obtaining an angle associated with the one or more imaging devices with respect to the anatomy; and
aligning the location sensor coordinate frame and the two-dimensional image data coordinate frame based on the angle.

11. (canceled)

12. (canceled)

13. The method of claim 1, wherein determining the transform between the location sensor coordinate frame and the two-dimensional image data coordinate frame further comprises generating a three-dimensional representation of the two-dimensional image data.

14. The method of claim 1, further comprising:
obtaining non-contrasted two-dimensional data from the one or more imaging devices at a time period after obtaining the two-dimensional image data;
determining an instrument shape; and
causing the non-contrasted two-dimensional data to be rendered on a display device with a representation of the first segment based on the instrument shape.

15. The method of claim 14, further comprising:
obtaining additional location sensor data of the instrument, the additional location sensor data being indicative of positions of the instrument moving within the anatomy over a second time period, wherein the determining of the instrument shape is based on the additional location sensor data.

16. The method of claim 14, further comprising identifying a second segment within the non-contrasted two-dimensional image data as corresponding to the instrument, wherein determining the instrument shape is based on the second segment.

17. A medical system that can augment a two-dimensional image with positional information of an instrument, the medical system comprising:
one or more imaging devices that generate two-dimensional image data corresponding to a location sensor coordinate frame; and
a memory that stores computer-executable instructions that, when executed by a control circuitry, cause the control circuitry to perform:
identify a first segment of an anatomy within the two-dimensional image data as corresponding to a part of the anatomy, the two-dimensional image data corresponding to a two-dimensional image data coordinate frame;
obtain location sensor data of the instrument from a location sensor, the location sensor data being indicative of positions of the instrument moving within the anatomy over a first time period;
use the location sensor data and the first segment of the anatomy to determine a transform between the location sensor coordinate frame and the two-dimensional image data coordinate frame;
determine an updated location of the instrument using additional location sensor data generated from the location sensor; and
cause data indicative of the updated location to be displayed within the two-dimensional image using the transform.

18. (canceled)

19. (canceled)

20. (canceled)

21. (canceled)

22. (canceled)

23. (canceled)

24. (canceled)

25. The medical system of claim 17, wherein determine the transform between the location sensor coordinate frame and the two-dimensional image data coordinate frame further comprises:
generate a kidney map using the location sensor data; and
register the two-dimensional imaging data and the kidney map.

26. The medical system of claim 25, wherein the computer-executable instructions further cause the control circuitry to perform:
obtain an angle associated with the one or more imaging devices with respect to the anatomy; and
align the location sensor coordinate frame and the two-dimensional image data coordinate frame based on the angle.

27. (canceled)

28. (canceled)

29. The medical system of claim 17, wherein determine the transform between the location sensor coordinate frame and the two-dimensional image data coordinate frame further comprises generate a three-dimensional representation of the two-dimensional image data.

30. The medical system of claim 17, wherein the computer-executable instructions further cause the control circuitry to perform:
obtain non-contrasted two-dimensional data from the one or more imaging devices at a time period after obtaining the two-dimensional image data;
determine an instrument shape; and
cause the non-contrasted two-dimensional data to be rendered on a display device with a representation of the first segment based on the instrument shape.

31. The medical system of claim 30, wherein the computer-executable instructions further cause the control circuitry to perform:
obtain additional location sensor data of the instrument, the additional location sensor data being indicative of positions of the instrument moving within the anatomy over a second time period, wherein determine the instrument shape is based on the additional location sensor data.

32. The medical system of claim 30, wherein the computer-executable instructions further cause the control circuitry to perform:
identify a second segment within the non-contrasted two-dimensional image data as corresponding to the instrument, wherein determining the instrument shape is based on the second segment.

33. A non-transitory computer readable storage medium having stored thereon instructions that, when executed, cause a processor of a device to at least:
obtain two-dimensional image data generated by one or more imaging devices of a medical system, the two-dimensional image data corresponding to a location sensor coordinate frame;
identify a first segment of an anatomy within the two-dimensional image data as corresponding to a part of the anatomy, the two-dimensional image data corresponding to a two-dimensional image data coordinate frame;
obtain location sensor data of an instrument from a location sensor, the location sensor data being indicative of positions of the instrument moving within the anatomy over a first time period;
use the location sensor data and the first segment of the anatomy, determining a transform between the location sensor coordinate frame and the two-dimensional image data coordinate frame;
determine an updated location of the instrument using additional location sensor data generated from the location sensor; and
cause data indicative of the updated location to be displayed within the two-dimensional image using the transform.

34. (canceled)

35. (canceled)

36. (canceled)

37. (canceled)

38. (canceled)

39. (canceled)

40. (canceled)

41. The non-transitory computer readable storage medium of claim 33, wherein determine the transform between the location sensor coordinate frame and the two-dimensional image data coordinate frame further comprises:
generate a kidney map using the location sensor data; and
register the two-dimensional imaging data and the kidney map.

42. The non-transitory computer readable storage medium of claim 41, wherein the instructions further cause the processor to:
obtain an angle associated with the one or more imaging devices with respect to the anatomy; and
align the location sensor coordinate frame and the two-dimensional image data coordinate frame based on the angle.

43. (canceled)

44. (canceled)

45. (canceled)

46. The non-transitory computer readable storage medium of claim 33, wherein the instructions further cause the processor to:
obtain non-contrasted two-dimensional data from the one or more imaging devices at a time period after obtaining the two-dimensional image data;
determine an instrument shape; and
causing the non-contrasted two-dimensional data to be rendered on a display device with a representation of the first segment based on the instrument shape.

47. The non-transitory computer readable storage medium of claim 46, wherein the instructions further cause the processor to:
obtain additional location sensor data of the instrument, the additional location sensor data being indicative of positions of the instrument moving within the anatomy over a second time period, wherein determine of the instrument shape is based on the additional location sensor data.

48. The non-transitory computer readable storage medium of claim 46, wherein the instructions further cause the processor to identify a second segment within the non-contrasted two-dimensional image data as corresponding to the instrument, wherein determining the instrument shape is based on the second segment.
